(19) United States
(12) Patent Application Publication
(10) Pub. No.: US 2021/0161779 A1
(43) Pub. Date: Jun. 3, 2021

(54) Hand Sanitizing Compositions

(71) Applicant: Carrie D. Alspaugh, M.D., P.A., Cary, NC (US)

(72) Inventors: James Andrew Alspaugh, II, Raleigh, NC (US); Carrie Deener Alspaugh, Raleigh, NC (US)

(21) Appl. No.: 17/176,644

(22) Filed: Feb. 16, 2021

Related U.S. Application Data
(63) Continuation of application No. 15/846,870, filed on Dec. 19, 2017.
Application No. 15/846,870 is a continuation of application No. PCT/US16/38932, filed on Jun. 23, 2016.
(60) Provisional application No. 62/183,254, filed on Jun. 23, 2015.

(51) Int. Cl. A61K 8/20; A61Q 17/02; A61K 8/36; A61K 8/49; A61Q 19/10; C01B 11/04; A61K 8/92; A61Q 17/00; A61K 8/46; C01B 13/10; A61K 8/29; A61K 8/27; A61Q 19/00; A61K 8/25; A61K 8/67; A01N 59/00; A61Q 17/04

(52) U.S. Cl. CPC A61K 8/20; A61K 2800/75; A61K 8/361; A61K 8/496; A61K 8/4966; A61Q 19/10; C01B 11/04; A61K 8/922; A61Q 17/005; A61K 8/466; C01B 13/10; A61K 8/29; A61K 8/27; A61Q 19/005; A61K 8/25; A61K 8/67; A01N 59/00; A61Q 17/04; A61K 2800/56; A61K 2800/522; A61K 2800/30; A61Q 17/02

(57) ABSTRACT

The present invention provides a sanitizing composition including a sanitizing effective amount of an agent selected from the group consisting of hypochlorous acid, sodium hypochlorite, calcium hypochlorite and combinations thereof in a carrier wherein the composition is substantially free of a drying alcohol. The present invention further provides methods of using the sanitizing compositions and articles of manufacture including the sanitizing compositions.

### RELATED APPLICATION DATA

The present application is a continuation application of U.S. patent application Ser. No. 15/846,870, filed Dec. 19, 2017, which is a bypass continuation application of International Application No. PCT/US16/38932, filed Jun. 23, 2016, which claims priority from and the benefit of U.S. Provisional patent Application No. 62/183,254, filed Jun. 23, 2015, the disclosures of which are hereby incorporated herein by reference in their entireties.

### FIELD

The present invention relates to sanitizing compositions that provide reduced skin irritation to subjects.

### BACKGROUND

Subjects with skin irritation are at increased risk for superficial microbial infections due to skin fissures and cracks on the skin surface that may result from the subject's condition. These changes in skin morphology represent an alteration in the skin barrier integrity, allowing a site of entry for surface microbes. Subjects with skin irritation, for example, eczema, are also very susceptible to agents that result in skin dryness exacerbating their condition and rendering them more susceptible to superficial microbial infections.

### SUMMARY

Embodiments of the present invention provide sanitizing compositions and methods of controlling microbial infections on the skin of a subject including applying the sanitizing compositions described herein to the skin of the subject. Embodiments of these sanitizing compositions and methods of using the same are environmentally safe and/or organic.

These sanitizing compositions of present invention include a sanitizing effective amount of an agent selected from the group consisting of hypochlorous acid, sodium hypochlorite, calcium hypochlorite and combinations thereof, wherein the composition is substantially free of a drying alcohol. The sanitizing compositions may also include a flowing agent. The sanitizing compositions may include (a) a sanitizing effective amount of an agent selected from the group consisting of hypochlorous acid, sodium hypochlorite, calcium hypochlorite and combinations thereof; (b) an oil phase; and (c) an aqueous phase, in a carrier wherein the composition is substantially free of a drying alcohol. In some embodiments, the sanitizing compositions also include a sun protecting agent and/or an insect repelling agent. The sanitizing compositions of the present invention provide antimicrobial activity in addition to enhanced moisturization, sun protection and/or insect repellant properties over conventional hand washing techniques and commercially available alcohol and non-alcohol based sanitizers.

Embodiments of the present invention also provide an article of manufacture including the sanitizing compositions of the present invention.

Additional embodiments of the present invention provide a method of making a hand sanitizing composition. In some embodiments, the method includes combining at least one of hypochlorous acid, sodium hypochlorite or calcium hypochlorite with an agent providing an oil phase and an agent providing an aqueous phase to form a hand sanitizing composition that is essentially free of a drying alcohol. In other embodiments, the method includes combining at least one of hypochlorous acid, sodium hypochlorite or calcium hypochlorite with a flowing agent to form a hand sanitizing composition that is essentially free of a drying alcohol.

Embodiments of the present invention provide a method of providing an environmentally-safe method for sanitizing a surface including applying the sanitizing composition of the present invention or an article of manufacture, such as a wipe, of the present invention to a surface. In some embodiments, the surface is the surface of an inanimate object. In some embodiments, the surface is a skin surface.

The present invention also provides protection for those subjects suffering from or at risk for skin irritation (such as a dermal inflammatory condition) due to hand washing by providing an alternative to hand washing with conventional soaps or cleansing with currently available alcohol and non-alcohol based sanitizers.

The foregoing and other aspects of the present invention are explained in greater detail as set forth below.

### BRIEF DESCRIPTION OF THE DRAWINGS

FIG. 1 presents results of antibacterial activity of hypochlorous acid-based cream against Staphylococcus aureus. Results of colony growth are shown in FIG. 1.

FIG. 2 presents results of antibacterial activity of hypochlorous acid-based cream against Staphylococcus aureus. Results of colony growth are shown in FIG. 2.

### DETAILED DESCRIPTION

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention pertains. The terminology used in the description of the invention herein is for the purpose of describing particular embodiments only and is not intended to be limiting of the invention. As used in the description of the invention and the claims set forth herein, the singular forms “a,” “an,” and “the” are intended to include the plural forms as well, unless the context clearly indicates otherwise. As used herein, the term “and/or” includes any and all combinations of one or more of the associated listed items.

All publications, patent applications, patents and other references cited herein are incorporated by reference in their entireties for the teachings relevant to the sentence and/or paragraph in which the reference is presented.

For purposes of this application, the chemical elements are identified in accordance with the Periodic Table of the Elements, CAS version, Handbook of Chemistry and Physics, 85th Ed., inside cover, and specific functional groups are defined as described therein. Additionally, general principles of organic chemistry, as well as specific functional moieties and reactivity, are described in Organic Chemistry, Francis A. Carey, McGraw-Hill, 2000, the contents of which are incorporated herein by reference.

### 1. Definitions

“Sanitizing” as used herein refers to anti-microbial, bactericidal, antiviral and/or disinfectant activity, including retarding the growth of at least one microorganism by any mechanism of action or system of activation. In general, the sanitizing products and methods of the present invention reduce the number of microbial contaminants (e.g., bacteria, fungi, viruses, and parasites) to safe levels as judged by public health requirements.

As used herein, the term “microorganism” refers to microscopic organisms that can exist as a single cell or cell clusters. Microorganisms that can be affected according to the present invention include, but are not limited to, bacteria, fungi, viruses, and parasites.

Bacterial infections that can be affected using the present invention can be caused by bacteria such as gram-negative bacteria. Examples of gram-negative bacteria include, but are not limited to, bacteria of the genera Salmonella, Escherichia, Klebsiella, Haemophilus, Pseudomonas, Proteus, Neisseria, Vibro, Helicobacter, Brucella, Bordetella, Legionella, Campylobacter, Francisella, Pasteurella, Yersinia, Bartonella, Bacteroides, Streptobacillus, Spirillum and Shigella. Furthermore, bacterial infections that can be treated using the sanitizing compositions of the present invention can be caused by gram-negative bacteria including, but not limited to, Escherichia coli, Pseudomonas aeruginosa, Neisseria meningitides, Neisseria gonorrhoeae, Salmonella typhimurium, Salmonella entertidis, Klebsiella pneumoniae, Haemophilus influenzae, Haemophilus ducreyi, Proteus mirabilis, Vibro cholera, Helicobacter pylori, Brucella abortis, Brucella melitensis, Brucella suis, Bordetella pertussis, Bordetella parapertussis, Legionella pneumophila, Campylobacter fetus, Campylobacter jejuni, Francisella tularensis, Pasteurella multocida, Yersinia pestis, Bartonella bacilliformis, Bacteroides fragilis, Bartonella henselae, Streptobacillus moniliformis, Spirillum minus and Shigella dysenteriae.

Bacterial infections that can be affected using the present invention can also be caused by bacteria such as gram-positive bacteria. Examples of gram-positive bacteria include, but are not limited to, bacteria of the genera Listeria, Staphylococcus, Streptococcus, Bacillus, Corynebacterium, Peptostreptococcus, and Clostridium. Furthermore, bacterial infections that can be treated using the sanitizing compositions of the present invention can be caused by gram-positive bacteria including, but not limited to, Listeria monocytogenes, Staphylococcus aureus, Streptococcus pyogenes, Streptococcus pneumoniae, Bacillus cereus, Bacillus anthracis, Clostridium botulinum, Clostridium perfringens, Clostridium difficile, Clostridium tetani, Corynebacterium diphtheriae and Peptostreptococcus anaerobius. In some embodiments, the gram-positive bacterium is methicillin-resistant Staphylococcus aureus.

Additional bacterial infections that can be affected using the present invention can be caused by bacteria in the genera including, but not limited to, Actinomyces, Propionibacterium, Nocardia and Streptomyces. Furthermore, bacterial infections that can be treated using the sanitizing compositions of the present invention can be caused by bacteria including, but not limited to, Actinomyces israeli, Actinomyces gerencseriae, Actinomyces viscosus, Actinomyces naeslundii, Propionibacterium propionicus, Nocardia asteroides, Nocardia brasiliensis, Nocardia otitidiscaviarum and Streptomyces somaliensis.

The effect on bacterial infections described herein can be bacteriocidal or bacteriostatic.

Mycobacterial infections that can be affected by the present invention can be caused by mycobacteria belonging to the mycobacteria families including, but not limited to, Mycobacteriaceae. Additionally, mycobacterial infections that can be treated by the sanitizing compositions of the present invention can be caused by mycobacteria including, but not limited to, Mycobacterium tuberculosis, Mycobacterium leprae, Mycobacterium avium-intracellulare, Mycobacterium kansasii, and Mycobacterium ulcerans.

Fungal infections that can be affected by the present invention can be caused by fungi belonging to the genera including, but not limited to, Aspergillus, Candida, Cryptococcus, Coccidioides, Tinea, Sporothrix, Blastomyces, Histoplasma, and Pneumocystis. Additionally, fungal infections that can be treated using the sanitizing compositions of the present invention can be caused by fungi including, but not limited to, Aspergillus fumigatus, Aspergillus flavus, Aspergillus niger, Aspergillus terreus, Aspergillus nidulans, Candida albicans, Coccidioides immitis, Cryptococcus neoformans, Tinea unguium, Tinea corporis, Tinea cruris, Sporothrix schenckii, Blastomyces dermatitidis, Histoplasma capsulatum, and Histoplasma duboisii.

Viral infections that can be treated using the sanitizing compositions of the present invention can be caused by viruses belonging to the viral families including, but not limited to, Flaviviridae, Arenaviradae, Bunyaviridae, Filoviridae, Poxviridae, Togaviridae, Paramyxoviridae, Herpesviridae, Picornaviridae, Caliciviridae, Reoviridae, Rhabdoviridae, Papovaviridae, Parvoviridae, Adenoviridae, Hepadnaviridae, Coronaviridae, Retroviridae, and Orthomyxoviridae. Furthermore, viral infections that can be treated using the sanitizing compositions of the present invention can be caused by the viruses including, but not limited to, Yellow fever virus, St. Louis encephalitis virus, Dengue virus, Hepatitis G virus, Hepatitis C virus, Bovine diarrhea virus, West Nile virus, Japanese B encephalitis virus, Murray Valley encephalitis virus, Central European tick-borne encephalitis virus, Far eastern tick-born encephalitis virus, Kyasanur forest virus, Louping ill virus, Powassan virus, Omsk hemorrhagic fever virus, Kumilinge virus, Absetarov anzalova hypr virus, Ilheus virus, Rocio encephalitis virus, Langat virus, Lymphocytic choriomeningitis virus, Junin virus, Bolivian hemorrhagic fever virus, Lassa fever virus, California encephalitis virus, Hantaan virus, Nairobi sheep disease virus, Bunyamwera virus, Sandfly fever virus, Rift valley fever virus, Crimean-Congo hemorrhagic fever virus, Marburg virus, Ebola virus, Variola virus, Monkeypox virus, Vaccinia virus, Cowpox virus, Orf virus, Pseudocowpox virus, Molluscum contagiosum virus, Yaba monkey tumor virus, Tanapox virus, Raccoonpox virus, Camelpox virus, Mousepox virus, Tanterapox virus, Volepox virus, Buffalopox virus, Rabbitpox virus, Uasin gishu disease virus, Sealpox virus, Bovine papular stomatitis virus, Camel contagious ecthyma virus, Chamios contagious ecthyma virus, Red squirrel parapox virus, Juncopox virus, Pigeonpox virus, Psittacinepox virus, Quailpox virus, Sparrowpox virus, Starlingpox virus, Peacockpox virus, Penguinpox virus, Mynahpox virus, Sheeppox virus, Goatpox virus, Lumpy skin disease virus, Myxoma virus, Hare fibroma virus, Fibroma virus, Squirrel fibroma virus, Malignant rabbit fibroma virus, Swinepox virus, Yaba-like disease virus, Albatrosspox virus, Cotia virus, Embu virus, Marmosetpox virus, Marsupialpox virus, Mule deer poxvirus virus, Volepox virus, Skunkpox virus, Rubella virus, Eastern equine encephalitis virus, Western equine encephalitis virus, Venezuelan equine encephalitis virus, Sindbis virus, Semliki forest virus, Chikungunya virus, O'nyong-nyong virus, Ross river virus, Parainfluenza virus, Mumps virus, Measles virus (rubeola virus), Respiratory syncytial virus, Herpes simplex virus type 1, Herpes simplex virus type 2, Varicella-zoster virus, Epstein-Barr virus, Cytomegalovirus, Human b-lymphotrophic virus, Human herpesvirus 7, Human herpesvirus 8, Poliovirus, Coxsackie A virus, Coxsackie B virus, ECHOvirus, Rhinovirus, Hepatitis A virus, Mengovirus, ME virus, Encephalomyocarditis (EMC) virus, MM virus, Columbia SK virus, Norwalk agent, Hepatitis E virus, Colorado tick fever virus, Rotavirus, Vesicular stomatitis virus, Rabies virus, Papilloma virus, BK virus, JC virus, B19 virus, Adeno-associated virus, Adenovirus (including serotypes 3, 7, 14, 21), Hepatitis B virus, Coronavirus, Human T-cell lymphotrophic virus, Human immunodeficiency virus, Human foamy virus, Influenza viruses, types A, B, C, and Thogotovirus.

Parasitic infections that can be affected by the present invention can be caused by parasites belonging to the genera including, but not limited to, Entamoeba, Dientamoeba, Giardia, Balantidium, Trichomonas, Cryptosporidium, Isospora, Plasmodium, Leishmania, Trypanosoma, Babesia, Naegleria, Acanthamoeba, Balamuthia, Enterobius, Strongyloides, Ascaradia, Trichuris, Necator, Ancylostoma, Uncinaria, Onchocerca, Mesocestoides, Echinococcus, Taenia, Diphylobothrium, Hymenolepsis, Moniezia, Dicytocaulus, Dirofilaria, Wuchereria, Brugia, Toxocara, Rhabditida, Spirurida, Dicrocoelium, Clonorchis, Echinostoma, Fasciola, Fascioloides, Opisthorchis, Paragonimus, and Schistosoma. Additionally, parasitic infections that can be treated using the sanitizing compositions of the present invention can be caused by parasites including, but not limited to, Entamoeba histolytica, Dientamoeba fragilis, Giardia lamblia, Balantidium coli, Trichomonas vaginalis, Cryptosporidium parvum, Isospora belli, Plasmodium malariae, Plasmodium ovale, Plasmodium falciparum, Plasmodium vivax, Leishmania braziliensis, Leishmania donovani, Leishmania tropica, Trypanosoma cruzi, Trypanosoma brucei, Babesia divergens, Babesia microti, Naegleria fowleri, Acanthamoeba culbertsoni, Acanthamoeba polyphaga, Acanthamoeba castellanii, Acanthamoeba astronyxis, Acanthamoeba hatchetti, Acanthamoeba rhysodes, Balamuthia mandrillaris, Enterobius vermicularis, Strongyloides stercoralis, Strongyloides fülleborni, Ascaris lumbricoides, Trichuris trichiura, Necator americanus, Ancylostoma duodenale, Ancylostoma ceylanicum, Ancylostoma braziliense, Ancylostoma caninum, Uncinaria stenocephala, Onchocerca volvulus, Mesocestoides variabilis, Echinococcus granulosus, Taenia solium, Diphylobothrium latum, Hymenolepis nana, Hymenolepis diminuta, Moniezia expansa, Moniezia benedeni, Dicytocaulus viviparous, Dicytocaulus filarial, Dicytocaulus arnfieldi, Dirofilaria repens, Dirofilaria immitis, Wuchereria bancrofti, Brugia malayi, Toxocara canis, Toxocara cati, Dicrocoelium dendriticum, Clonorchis sinensis, Echinostoma, Echinostoma ilocanum, Echinostoma jassyenese, Echinostoma malayanum, Echinostoma caproni, Fasciola hepatica, Fasciola gigantica, Fascioloides magna, Opisthorchis viverrini, Opisthorchis felineus, Opisthorchis sinensis, Paragonimus westermani, Schistosoma japonicum, Schistosoma mansoni, Schistosoma haematobium and Schistosoma haematobium.

“Controlling” as used herein refers to retarding, preventing or inhibiting microbial infection. Controlling microbial infections means that a microbial infection does not develop, does not spread and/or does not worsen where microbial population growth is eliminated, inhibited, contained, and/or maintained at a level such that the level of microbial activity does not result in symptoms of illness.

“Retarding the growth” refers to reducing, delaying and/or hindering activity contributing to the growth of the microorganism. “Reducing” or “reduce” refers to a decrease or diminishment in the specified activity of at least about 10%, 15%, 20% 25%, 35%, 40%, 50%, 60%, 75%, 80%, 85%, 90%, 95% or more. In some embodiments, the reduction results in little or essentially no detectible activity (at most, an insignificant amount, e.g., less than about 10% or even 5%). In any case, the retarded growth is beneficial to the subject.

As used herein, the term “bacteriocidal” refers to killing the bacteria.

As used herein, the term “bacteriostatic” refers to inhibiting bacterial growth without killing.

As used herein, the term “carrier” refers to carriers, diluents, or excipients that are compatible with the other ingredients of the composition in that it can be combined with the active ingredients and/or compositions of the present invention without eliminating the desired activity of the active ingredients of the compositions, and is suitable for use in subjects as provided herein without undue adverse side effects (such as toxicity, irritation, allergic response, death and the like). Side effects are “undue” when their risk outweighs the benefit provided by the pharmaceutical composition.

“Dermatologically acceptable carrier” as used herein refers to a carrier that is suitable for topical application to the keratinous tissue, may have desirable aesthetic properties, is compatible with the active ingredients of the present invention and potentially other components, and will not cause any undesirable safety or toxicity concerns. The dermatologically acceptable carrier may be in a wide variety of forms such as, for example, simple solutions (water-based or oil-based), solid forms (e.g. gels or sticks) and emulsions. Carriers may include, but are not limited to, liquids and sterile liquids, such as water and oils, including those of petroleum, animal, vegetable or synthetic origin, such as jojoba oil, coconut oil, aloe vera oil, peanut oil, soybean oil, mineral oil, sesame oil and the like, polyethylene glycols, glycerine, propylene glycol or other synthetic solvents.

As used herein, the term “effective amount” refers to an amount of an ingredient or composition that is sufficient to produce the desired effect, which can be a therapeutic effect or a practically significant effect. The effective amount will vary with the application for which the ingredient or composition is being employed, the microorganism and/or the age and physical condition of the subject, the severity of the condition, the duration of the treatment, the nature of any concurrent treatment, the carrier used, and like factors within the knowledge and expertise of those skilled in the art. An appropriate “effective amount” in any individual case can be determined by one of ordinary skill in the art by reference to the pertinent texts and literature and/or by using routine experimentation. (See, for example for pharmaceutical applications, Remington, The Science And Practice of Pharmacy (9th Ed. 1995).

“Skin” as used herein refers to the epidermis, dermis, and hypodermis (i.e., subcutis), and also includes the mucosa and skin adenexa, particularly hair follicles, hair roots, hair bulbs, the ventral epithelial layer of the nail bed (lectulus) as well as sebaceous glands and perspiratory glands (eccrine and apocrine).

“Sun protecting agent” as used herein refers to an agent that reflects or absorbs at least some of the sun's ultraviolet radiation. The sun protecting agent may be a physical protecting agent or a chemical protecting agent.

“Ultraviolet” radiation as used herein refers to radiation from the sun covering wavelengths in the range 100-400 nm and is divided into three bands: UVA (315-400 nm), UVB (280-315 nm) and UVC (100-280 nm).

As used herein, “sun protection factor” or “SPF” refers to a rating measure of the fraction of sunburn-producing UV rays blocked. For example, “SPF 15” means that 1/15th of the burning radiation will reach the skin under conditions specified for administration of the sun protecting agent.

“Protection Grade” system as used herein refers to a rating measure based on the persistent pigment darkening (PPD) reaction, which uses UVA radiation to cause a persistent darkening or tanning of the skin. Theoretically, a sunscreen with a PPD rating of 10 should allow a person 10 times as much UVA exposure as would be without protection. According to the Japan Cosmetic Industry Association, PA+ corresponds to a UVA protection factor between two and four, PA++ between four and eight, and PA+++ more than eight.

“Emollient” as used herein refers to an agent that is capable of maintaining or improving the moisture level and/or compliance of the skin.

“Essential oil” as used herein refers to typically concentrated, hydrophobic liquids containing fragrant molecules from plants. Essential oils include those derived from berries, allspice, juniper, seeds, almond, anise, celery, cumin, nutmeg oil, bark, cassia, cinnamon, sassafras, wood, camphor, cedar, rosewood, sandalwood, agarwood, rhizome, galangal, ginger, leaves, basil, bay leaf, cinnamon, common sage, eucalyptus, lemon grass, melaleuca, oregano, patchouli, peppermint, pine, rosemary, spearmint, tea tree, thyme, wintergreen, resin, frankincense, myrrh, flowers, cannabis, chamomile, clary sage, clove, scented geranium, hops, hyssop, jasmine, lavender, manuka, marjoram, rose, rosemary, basil, lemon grass, ylang-ylang, peel, bergamot, grapefruit, lemon, lime, orange, tangerine, root, valerian, mango, or the like. Embodiments of the present invention may include one or more of these essential oils.

As used herein, “insect repelling agent” refers to an agent that repels insects or other pest arthropods and that can be applied to skin or another surface.

“Skin irritation” as used herein refers to dry, itchy, red, bumpy, scaly and/or swollen skin. The skin irritation may result from a dermal inflammatory condition.

“Dermal inflammatory condition” as used herein refers to skin diseases that are triggered and maintained by aberrant responses of the cells of the skin immune system. Examples of dermal or skin inflammatory conditions include, but are not limited to, dermatitis (such as allergic contact dermatitis, seborrheic dermatitis or radiation dermatitis), psoriasis, pruritus, eczema, urticaria, angiodema and combinations thereof.

“Environmentally safe”, “environmentally preferred”, “environmentally friendly”, or “green”, as used herein refer to the quality of not being harmful to plants and/or animals when exposure is under normal conditions or deemed to inflict reduced, minimal, or no harm upon ecosystems and/or the environment.

“Organic” as used herein refers to compliance with the requirements of the United States Department of Agriculture's (USDA's) National Organic Program (NOP) for products labeled as “organic” or “made with organic.”

“Substantially free” as used herein refers to containing less than about 0.1, 0.5, or 1% by weight of the substance, based upon 100% weight of total composition. It is possible that trace amounts of the agent may be present in the composition.

As used herein, the term “combining” refers to the mixing or admixing of ingredients in a composition or formulation.

### 2. Compositions

Sanitizing compositions of the present invention comprise, consist essentially of or consists of: (a) a sanitizing effective amount of an agent selected from the group consisting of hypochlorous acid, sodium hypochlorite, calcium hypochlorite and combinations thereof; (b) an oil phase; and (c) an aqueous phase, in a dermatological carrier wherein the composition is substantially free of a drying alcohol (such as ethyl alcohols, such as ethanol, and isopropanol).

Sanitizing compositions of the present invention also comprise, consist essentially of or consists of (a) a sanitizing effective amount of an agent selected from the group consisting of hypochlorous acid, sodium hypochlorite, calcium hypochlorite and combinations thereof; and (b) a flowing agent, wherein the composition is substantially free of a drying alcohol.

In some embodiments, the oil phase comprises fats and/or oils. In particular embodiments, the oil phase comprises an emollient. In some embodiments, the emollient is selected from the group consisting of petrolatum, petrolatum based oils, vegetable based oils, mineral oils, natural or synthetic oils, fatty esters, glycerol esters, propylene glycol esters, alkoxylated carboxylic acids, alkoxylated alcohols, fatty alcohols, fatty acids, and combinations thereof. In further embodiments, the emollient is at least one of squalane, castor oil, odorless mineral spirits, sweet almond oil, avocado oil, calophyllum oil, ricin oil, vitamin E acetate, hydrogenated vegetable oil, beeswax, jojoba oil, lanolin, almond oil, olive oil, shea butter, coconut oil, wheat germ oil, aloe vera extract, grapeseed oil, tea tree oil, soybean oil, mint oil, rosemary oil, germanium oil, vegetable oil, cod liver oil, mineral oil, camphor oil, cade oil, eucalyptol, clove oil, aloe vera, polybutene, cetyl alcohol, dimethicone, cyclopentasiloxane, dimethiconol, dimethicone crosspolymer, trisiloxane, dimethylopolysiloxane, cyclomethicone, isopropyl palmitate, octyl palmitate, isopropyl myristate, hexadecyl stearate, butyl stearate, decyl oleate, and acetyl glyceride.

In some embodiments, the aqueous phase comprises water, glycerin, or a combination thereof. The water may be sterile, injection grade (WFI) (highly purified waters containing less than 10 CFU/100 ml of aerobic bacteria), deionized, demineralized, potable, buffered and/or electrolyzed water. In some embodiments, the final water used in the sanitizing compositions is the product of a dilute solution of sodium chloride in water that has been electrolyzed to generate solutions of small quantities of hypochlorous acid as well as small quantities of ozone. These small quantities of hypochlorous acid and ozone can be removed by heating.

In embodiments including a flowing agent, the flowing agent promotes the flowability of the product. A flowing agent may prevent agglomeration and clumping and/or prevent bridge formation. In embodiments of the present invention, the flowing agent may be selected from magnesium stearate, calcium stearate, zinc state, stearic acid and fumed silica. In particular embodiments, the flowing agent is hydrophilic fumed silica. The fumed silica may be a pharmaceutical grade product. The fumed silica may be obtained from a commercial supplier such as the fumed silica sold under the brand name AEROSIL® or AEROPERL®. The fumed silica may be present in an amount ranging from 0.1% to 15%, in some embodiments, up to 10%, in some embodiments, up to 5%, with all values leading up to the maximum value being included herein.

In some embodiments, the sanitizing composition further includes a component selected from the group consisting of a humectant, a lubricant, a carrier, a pharmaceutically active agent, a color, a fragrance, a preservative, an antioxidant, a chelator, a neutralizer, an amino acid, an anti-inflammatory agent, an anti-irritant, a binders, a stabilizer, an emollient, an emulsifier, a surfactant, a foaming agent, an essential oil, a plant/botanical extract, a skin conditioning agent, a gelling agent, a humectant, a vitamin, a mineral, a viscosity enhancer, a pH modifier, a protein, a peptide, a tactile enhancer, and combinations thereof.

In particular embodiments, the sanitizing composition includes at least one of a viscosity enhancer, a pH modifier, a humectant (such as hydroxyethyl urea, agarose, urea, sodium PCA, arginine PCA, fructose, glucose, glutamic acid, glycerine, honey, lactose, maltose, polyethylene glycol, sorbitol and mixtures thereof), an antioxidant (ascorbic acid (vitamin C), tocopherols (vitamin E), alpha lipoic acid, grape seed extract, green tea extract, L-ergothioneine and resveratrol), an anti-inflammatory agent (non-steroidal anti-inflammatory agents such as ibuprofen, acetaminophen, ketoprofen, indomethacin, aspirin, and the like, and corticosteroids) a skin conditioning agent (such as oils described herein, fatty acids, glycol fatty acids, vitamins, sugars, glycerin, propylene glycol, sorbitols, and polyethylene glycols) an essential oil, a vitamin and a mineral (selenium (Se), zinc (Zn), potassium (K), calcium (Ca), magnesium (Mg), sulfate (SO4), bromide (Br), manganese (Mn), copper (Cu), sulfur (S), silica (SiO2), iron (Fe), bicarbonate and tellurium (Te), sodium (Na) and chloride (Cl) including derivatives, oxides and combinations thereof).

According to further embodiments of the present invention, the sanitizing compositions further comprise a sun protecting agent. The sun protecting agent may be selected from the group consisting of zinc oxide, titanium dioxide, octocrylene, octyl salicylate, avobenzone, ecamsule, octyl triazone, amiloxate, diethylhexyl butamido trizone, enzacamene, drometrizole trisiloxane, bemotrizinol, bisoctrizole and combinations thereof. In some embodiments, the sun protecting agent is present in an amount from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 or 25% total weight of the sanitizing composition. In particular embodiments, the sun protecting agent is zinc oxide and/or titanium dioxide. The zinc oxide and/or titanium oxide can be U.S. Pharmacopeial Convention (USP) food grade quality and/or generally recognized as safe (GRAS) by the U.S. Food and Drug Administration. In some embodiments, the zinc oxide and/or titanium oxide is present in the sanitizing compositions as nanoparticles having a particle size less than 100 nm, between 1 and 100 nm, between 1-50 nm, between 1-30 nm, between 1-20 nm and 1-10 nm, with particle size and measurements of the same being understood by one of ordinary skill in the art. In some embodiments, the zinc oxide and/or titanium oxide is present in the sanitizing compositions as non-nanoparticles having a particle size greater than 100 nm. In some embodiments, the sanitizing compositions include the zinc oxide and titanium dioxide in mixtures having a ratio of from 4:1 to 8:1.

In particular embodiments, the sun protecting agent is substantially free of para-aminobenzoic acid (PABA), paraben and/or phthalate.

In some embodiments, the sanitizing composition has a sun protection factor (SPF) of 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105 or 110. In some embodiments, the sanitizing composition has an SPF in a range from about 8 to 110, 8 to 70, 8 to 50, 8 to 30, 8 to 15. In some embodiments, the sanitizing composition has an SPF of 15. In some embodiments, the sanitizing composition has an SPF of 30. In some embodiments, the sanitizing composition has an SPF of at least 50. In some embodiments, the sanitizing composition has a protection grade of PA+. In some embodiments, the sanitizing composition has a protection grade of PA++. In some embodiments, the sanitizing composition has a protection grade of PA+++.

In embodiments including the sun protecting agent, subjects using the sanitizing composition may decrease their risk of skin cancer, and in particular, basal cell cancer, squamous cell cancer and/or melanoma as well as decrease their risk of pre-cancer growths, e.g., actinic keratoses.

Further, embodiments including the sun protecting agent may provide subjects using the sanitizing composition the benefit of preventing, reducing, delaying and/or minimizing the appearance of photo-aging, e.g., lentigines or liver spots.

Additionally, embodiments including the sun protecting agent may assist with preventing, reducing, delaying and/or minimizing the appearance of fine lines and/or wrinkles.

Embodiments of the present invention further provide sanitizing compositions comprising an insect repelling agent. In some embodiments, the insect repelling agent comprises at least one of citronella, citronella oil, lemon grass oil, limonene, rosemary oil, eucalyptus oil, lemon juice, tea tree oil, mint oil, soybean oil, rosemary oil, geranium oil, geraniol, vegetable oil, camphor oil and eucalyptol.

According to some embodiments of the present invention, the sanitizing composition comprises a component selected from the group consisting of a viscosity enhancer, a pH modifier, a humectant, a skin conditioning agent, vitamins, and combinations thereof. Viscosity enhancers, such as thickeners, may be added to the sanitizer to increase the viscosity of the sanitizer. Suitable viscosity enhancers include clays and derivatives thereof, silicates, silicas and derivatives thereof, and combinations thereof. Suitable clays and derivatives thereof include, but are not limited to, kaolin, bentonite and derivatives thereof such as quaternium-18 bentonite, hectorite and derivatives thereof such as quaternium-18 hectorite, montmorillonite, and combinations thereof. Suitable silicates include, but are not limited to, magnesium aluminum silicate, sodium magnesium silicate, lithium magnesium silicate, tromethamine magnesium aluminum silicate, and combinations thereof. Suitable silicas and derivatives thereof include, but are not limited to, silica, hydrated silica, hydrophobic silica, silica silylate, silica methyl silylate, colloidal silicone dioxide, fumed silica, and combinations thereof. Other examples of suitable viscosity enhancers include mica, polyolefin resins, lipophilic/oil thickeners, ethylene/vinyl acetate copolymers, polyethylene, cetyl hydroxy ethyl cellulose, hydroxyethylcellulose, hydroxypropyl methylcellulose, hydroxypropylcellulose, other organically modified celluloses, PVP/decane copolymer, PVM/MA decadiene crosspolymer, PVP/eicosene copolymer, PVP/hexadecane copolymer, butylated PVP, carbomers, acrylic based thickeners, polyethylene glycol 600, polyethylene glycols, myristyl alcohol, cetyl alcohol, stearyl alcohol, behenyl alcohol, PEG-150 distearate, PEG-175 diisostearate, polyglyceryl-10 behenate/eicosadioate, disteareth-100 IPDI, polyacrylamidomethylpropane sulfonic acid, silicone crosspolymers, polyamide blends, and combinations thereof.

In general, the pH of the sanitizer may be controlled to be within any desired range. For use on a human body, it is typically desirable to have a sanitizer with a neutral pH. If necessary, various pH modifiers may be utilized in the sanitizer to achieve the desired pH level. Any suitable acid or alkali material may be used as a pH modifier. In particular, useful pH-adjusting agents include acids, such as hydrochloric acid, bases or buffers, such as sodium lactate, sodium acetate, sodium phosphate, sodium citrate, sodium borate, or sodium gluconate. In some embodiments, the pH modifier is one of lactic acid, citric acid, sodium citrate, glycolic acid, succinic acid, phosphoric acid, monosodium phosphate, disodium phosphate, oxalic acid, dl-malic acid, calcium carbonate, sodium hydroxide, sodium carbonate, sodium hydrogen carbonate, or ammonium hydrogen carbonate. In some embodiments, the pH modifier is citric acid or lactic acid.

In particular embodiments, the sanitizing compositions have a pH in a range of about 4.0 to about 8.0 at room temperature, and in some embodiments, 5.0-7.0.

Suitable humectants include glycerin, polymeric glycols such as polyethylene glycol and polypropylene glycol, mannitol and sorbitol.

Suitable vitamins include vitamin E, K, C, D, A, B and derivatives thereof.

In particular embodiments, the sanitizing composition is substantially free of a non-naturally occurring fragrance where less than about 0.1, 0.5, or 1% by weight of a non-naturally occurring fragrance, based upon 100% weight of total composition, is present. “Non-naturally occurring” used herein is meant ingredients, or components, that do not occur in nature. In some embodiments, it is possible that trace amounts of a non-naturally occurring fragrance may be present in the sanitizing compositions.

In other embodiments, the sanitizing composition is substantially free of a drying alcohol where less than about 0.1, 0.5, or 1% by weight of a drying alcohol, based upon 100% weight of total composition, is present. In some embodiments, the sanitizing composition is substantially free of methanol, ethanol (ethyl alcohol), propanol, isopropyl alcohol, butanol, t-butanol, 2-butanol, pentanol, hexanol, and combinations thereof.

In particular embodiments, the sanitizing compositions provide greater stability or shelf life than conventional sanitizing agents, particularly those including a drying alcohol. The sanitizing compositions of the present invention may provide effective antimicrobial activity for a period longer than that provided by conventional sanitizing agents, particularly those including a drying alcohol.

According to embodiments of the present invention, the sanitizing compositions may be formulated as a gel, lotion, cream, liquid, spray, aerosol, foam or mousse. In particular embodiments, the sanitizing composition is a lotion or cream. In particular embodiments, the sanitizing composition is a lotion or cream including a sun protecting agent and/or an insect repelling agent.

In particular embodiments of the present invention, the sanitizing composition is suitable for application to a surface in need of sanitation and/or disinfection. In some embodiments, the surface is an inanimate object. In some embodiments, the surface is a skin surface. In some embodiments, the sanitizing composition is applied anywhere on a subject's body including a subject's hands, head, face, neck, torso, back, arms and legs.

Suitable subjects according to the present invention include, but are not limited to, avian, mammalian and plant subjects. Illustrative avians according to the present invention include chickens, ducks, turkeys, geese, quail, pheasant, ratites (e.g., ostrich) and domesticated birds (e.g., parrots and canaries), and birds in ovo.

Mammals of the present invention include, but are not limited to, canines, felines, bovines, caprines, equines, ovines, porcines, rodents (e.g. rats and mice), lagomorphs, primates (including non-human primates), humans, and the like. According to some embodiments of the present invention, the mammal is a non-human mammal (living or deceased). In some embodiments, the mammal is a human subject (living or deceased). Human subjects of both genders are suitable subjects according to the present invention. Further, the subjects relevant to this invention may be male or female and may be any age such as less than 12 months to over 100 including newborns, infants, juveniles, adolescents, teenagers, adults and geriatrics. Subjects relevant to this invention further include any species and may be of any race or ethnicity, including, but not limited to, Caucasian, African-American, African, Asian, Hispanic, Indian, etc., and combined backgrounds. Particularly relevant subjects to this invention are subjects who suffer from skin irritation or are susceptible to skin irritation. Particular subjects are those who have dry skin as well as those who have eczema or are susceptible to eczema.

The present invention can also be carried out on animal subjects, particularly mammalian subjects such as mice, rats, dogs, cats, livestock and horses for veterinary purposes, and for screening and development purposes.

Embodiments of the present invention also provide articles of manufacture including the sanitizing compositions of the present invention. Articles of manufacture as used herein relate to products capable of being used or worn and which have the potential for contact with a microorganism.

Embodiments of the present invention also provide that the sanitizing compositions may be used in combination with a product, such as a personal care product. In particular, the sanitizing compositions may be incorporated into or onto a substrate, such as a wipe substrate, an absorbent substrate, a fabric or cloth substrate, a tissue substrate, or the like. The sanitizing compositions may be incorporated into personal care products, such as wipes, towelettes, absorbent articles, bath tissues, cloths, and the like. For example, the sanitizing compositions can be incorporated into the substrate by being applied to a substrate, for example, by spraying, coating, dipping, etc. The sanitizing compositions may be applied to the surface of a substrate or application may result in integration of the sanitizing compositions into the substrate.

According to embodiments of the present invention, the sanitizing compositions may be incorporated into wipes such as wet wipes, hand wipes, face wipes, cosmetic wipes, towelettes and the like, or absorbent articles, such as diapers, training pants, adult incontinence products, feminine hygiene products, and the like, and combinations thereof.

Embodiments of the present invention also provide methods of making sanitizing compositions comprising combining at least one of hypochlorous acid, sodium hypochlorite or calcium hypochlorite with an agent providing an oil phase and an agent providing an aqueous phase to form a sanitizing composition that is essentially free of a drying alcohol. In some embodiments, each active agent may comprise a lower limit ranging from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, and 10% to an upper limit ranging from about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 and 99.9% by weight of the composition.

### 3. Methods of Use

In addition to the benefits and methods of using the sanitizing compositions of the present invention as described above, embodiments according to the present invention further provide methods of sanitizing a surface comprising applying the sanitizing compositions or a wipe comprising the sanitizing compositions to a surface thereby reducing or retarding the growth of a microorganism. Microorganisms that can be affected by the methods of the present invention include the microorganisms described above. In particular embodiments, the microorganisms include Staphylococcus aureus, methicillin-resistant Staphylococcus aureus, Enterococcus faecalis VRE, Escherichia coli, Acinetobacter baumannii, Bacteroides fragilis, Candida albicans, Enterobacter aerogenes, Enterococcus faecium VRE-MDR, Haemophilus influenzae, Klebsiella oxytoca MDR, Klebsiella pneumoniae, Micrococcus luteus, Proteus mirabilis, Pseudomonas aeruginosa, Serratia marcescens, Staphylococcus epidemidis, Staphylococcus haemolyticus, Staphylococcus hominis, Staphylococcus saprophyticus, Staphylococcus pyogenes, and Aspergillus niger.

In some embodiments, the surface is an inanimate object such as furniture (stationary and non-stationary), appliances, fixtures, toys, tools, medical instruments, etc. Objects found in private environments such as homes; public environments such as schools, office buildings, libraries, parks, entertainment facilities, shopping facilities, etc.; and health care settings such as hospitals, clinics, physician's offices, school infirmaries, etc. are settings that contain objects providing a surface for application of the sanitizing compositions or a wipe comprising the sanitizing compositions. In some embodiments, the surface is a skin surface. In still other embodiments, the sanitizing composition or wipe is applied anywhere on a subject's body including a subject's hands, head face, neck, torso, back, arms and legs. In some embodiments, the method is non-irritating to the skin surface. In some embodiments, the subject has a dermal inflammatory condition or is susceptible to a dermal inflammatory condition. In some embodiments, the subject has dry skin or is susceptible to dry skin. In still other embodiments, the subject has eczema or is susceptible to eczema.

In further embodiments, the methods provide protection against sun ultraviolet (UV) radiation when applied to a skin surface.

According to other embodiments, the methods provide insect repellant activity.

In particular embodiments, an environmentally safe, environmentally preferred, environmentally friendly, or green method for sanitizing a surface is provided.

The present invention will now be described with reference to the following examples. It should be appreciated that these examples are for the purpose of illustrating aspects of the present invention, and do not limit the scope of the invention as defined by the claims.

### EXAMPLES

### Example 1

Compositions (weight percent/100% total)

### Composition 1

Agent: 1-99.8% hypochlorous acid
Oil phase: 0.1-25% emulsifying wax
0.1-25% jojoba oil
0.1-25% cetyl alcohol
0.1-25% shea butter
0.1-25% dimethicone
   Water phase: 0.1-80% water
0.1-25% glycerine
   pH: 4-7

### Composition 2

Agent: 1-99.8% hypochlorous acid
Oil phase: 0.1-25% emulsifying wax
0.1-25% jojoba oil
0.1-25% cetyl alcohol
0.1-25% shea butter
0.1-25% dimethicone
   Water phase: 0.1-80% water
0.1-25% glycerine
   Carriers/buffer: 0.1-15% succinic acid
0.1-15% citric acid
   pH: 4-7

### Composition 3

### See Compositions 1 or 2

Sun protecting agent: 0.1-15% zinc oxide

### Composition 4

### See Compositions 1 or 2

Sun protecting agent: 0.1-15% zinc oxide
Sun protecting agent: 0.1-15% titanium dioxide

### Composition 5

Agent: 1-99.8% hypochlorous acid
Flowing agent: 0.1-15% fumed silica
pH: 4-7

### Composition 6

### See Composition 5

Sun protecting agent: 0.1-15% zinc oxide

### Composition 7

### See Composition 5

Sun protecting agent: 0.1-15% zinc oxide
Sun protecting agent: 0.1-15% titanium dioxide

### Example 2

### Method of Making

The components of the oil phase are combined and mixed in one receptacle. The components in the water phase are combined and mixed in another receptacle. Where appropriate, the components are combined and mixed in a single receptacle. The components are heated to about 75-80° C. Where the components are provided in two receptacles, the contents of each receptacle are combined and further blended until smooth and homogenous. The temperature is lowered as necessary to prevent evaporation, destabilization, etc. In some instances, a sun protecting agent, insect repelling agent, vitamin and/or fragrance is added. The contents are mixed until smooth and homogenous. The composition is cooled and ultimately poured into a container allowing for application to a subject.

For compositions including a flowing agent, these compositions can be prepared using a cold emulsion process where it is not necessary to heat the components. Instead, the compositions can be prepared at ambient temperatures or less.

### Example 3

### Antimicrobial Activity

### ASTM E2315 Time Kill Assay for Antimicrobial Agents

In this method, a sample of the sanitizing compositions is inoculated with a suspension of a representative test organism. After a series of pre-selected exposure times, a sample is removed, neutralized and quantitatively assayed for surviving test organism. The resulting plates are incubated, the survivors are enumerated and percent and log 10 reductions are determined for each time point as compared to a population control. Common test organisms used to evaluate hand sanitizers as recommended by the Tentative Final Monograph include, but are not limited to: Staphylococcus aureus, Acinetobacter baumanii, Klebsiella pneumoniae, Streptococcus pyogenes, Pseudomonas aeruginosa, Burkholderia cepacia, and Serratia marcesens.

### ASTM E1052 Standard Test Method for Efficacy of Antimicrobial Agents Against Viruses in Suspension

An aliquot of the sanitizing compositions is inoculated with the test virus and held for the requested exposure time(s). At each pre-determined exposure time an aliquot is removed, neutralized by serial dilution, and assayed for viral infectivity by an assay method specific for the test virus. Appropriate virus, sanitizing composition cytotoxicity, and neutralization controls are run concurrently. The percent and log reduction in viral infectivity are calculated as compared to the corresponding virus control.

### Minimum Inhibitory Concentration (MIC) and Minimum Bactericidal Concentration (MBC) Tests

A series of sanitizing composition dilutions is placed into a 96-well plate. The wells containing the various product concentrations are then inoculated with test organisms of interest and the well plate is incubated. Following incubation, the wells are evaluated for presence or absence of the test organism. These data provide useful information related to the minimum inhibitory concentration or minimum bactericidal concentration of the sanitizing compositions.

### Glove Juice or Sterile Bag Procedure

An in vivo portion of efficacy testing is carried out on subjects using the sanitizing compositions by using a bacterium, such as Serratia marcescens, which is inoculated onto the hands of subjects and then recovered by massaging the hand(s) in a broth filled, sterile glove or bag. A quantity of liquid is removed, diluted and plated.

### Example 4

### Initial Evaluation of Antibacterial Activity of Hypochlorous Acid-Based Cream Experimental

A suspension of Staphylococcus aureus was made in sterile PBS with a target concentration of 10³ CFU/ml. Next, 2 ml of hand cream (or PBS control) was then added to tube 1. Serial 2-fold dilutions of the suspension were made into PBS (1:2, 1:4, 1:8, etc.), such that 1 ml of solution was left in each tube. Next, 1 ml bacterial suspension was added to each tube and incubated for 30 minutes at RT. Then, from each tube, 0.2 ml of treated cell suspension was plated to YT agar plates (a non-selective medium that supports S. aureus growth). The plates were incubated at 37° C. for 16 hours.

### Results

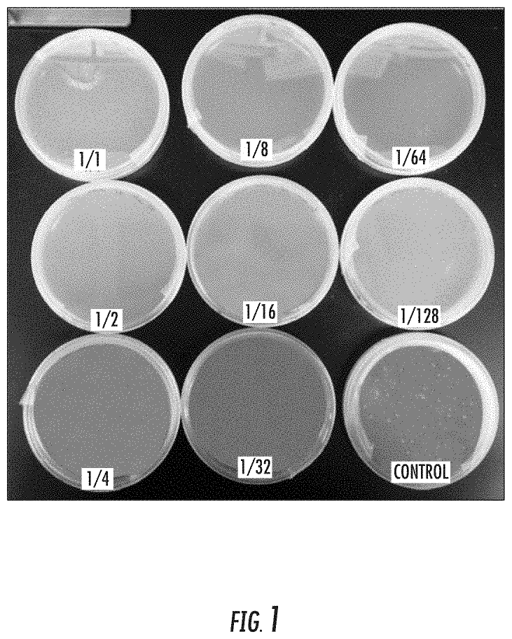

Following incubation at 37° C. for 16 hours, the plates were examined for colony growth. The results are shown in FIG. 1. Approximately 45 colonies appeared on the control plate containing no hand cream. No colony growth was observed on any plate with serial dilutions of hand cream up to a dilution 1/128.

### Example 5

### Evaluation of E-Water/Buffer

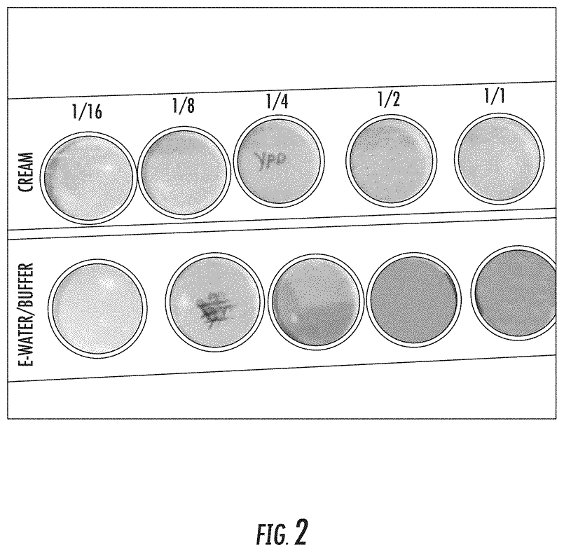

S. aureus with varying dilutions of E-water/buffer, as well as the cream, were made. Serial dilutions of 1/1, 1/2, 1/4, 1/8 and 1/16 were plated and examined for colony growth. No colonies of S. aureus were observed except for one colony on the 1/16 dilution. The cream appeared clear for the 1/1 and 1/2 dilutions. The results are shown in FIG. 2.

### Example 6

### Evaluation of UV Protection

An amount of the sanitizing composition corresponding to 1.3 mg/cm² is applied to 4 PMMA plates (Helioplate PMMA plates SB6, HelioScreen, Criel, Lot 803UK). The test product is applied by “spotting” the product on each plate and rubbing with a finger tip saturated with the test product for approximately one minute, then allowing equilibration in the dark for at least 30 minutes at 25° C.±2°. A solar simulator (Model LS1000-4S-009, Solar Light Company, Philadelphia) that complies with Colipa specifications is used to irradiate the plates with a series of 4 UV doses (11, 21, 32 and 43 J/cm²) and a calibrated UV-2000 Sunscreen Analyzer (Model UV-2000S, Labsphere, North Sutton, N.H.) that complies with Colipa specifications is used to measure the sunscreen absorbance spectra on each plate, before UV irradiation and after each UV dose.

### Estimated SPF

The initial SPF is determined for each plate, from the absorbance spectrum before irradiation, using the Sayre equation:

$$SPF = \frac{{\sum }_{290}^{400} {I}_{0 λ} {s}_{λ} Δ λ}{{\sum }_{290}^{400} {I}_{0 λ} {T}_{λ} {s}_{λ} Δ λ}$$

here:
I_{0λ}=spectral irradiance of the irradiation lamp
Sλ=The Mckinlay-Diffey erythemal effectiveness spectrum
T_{λ}=The mean initial transmission spectrum*
Δ_{λ}=the wavelength interval
*Transmission=10^{-A}, where A is absorbance.
To calculate the estimated overall SPF, the transmitted UV dose vs. applied UV dose is measured in MEDS for each plate, and the graph of mean applied UV dose (x) vs. mean transmitted UV dose (y) is generated. Then a least-squares curve fit of applied UV dose vs. transmitted UV dose provides a power curve equation in the form:

y=αx^{β}

The overall SPF is defined as the mean applied UV dose when the mean transmitted UV dose reaches 1 MED, and is analogous to the SPF measured on human subjects. Thus, the calculated SPF is given by: SPF=(1/α)^{(1/β)}.

The foregoing is illustrative of the present invention, and is not to be construed as limiting thereof. The invention is defined by the following claims, with equivalents of the claims to be included therein.

What is claimed is:

1. A method of controlling microbial infections on the skin of a subject, the method comprising applying to the skin of the subject a sanitizing composition comprising:
(a) a sanitizing effective amount of an agent selected from the group consisting of hypochlorous acid, sodium hypochlorite, calcium hypochlorite and combinations thereof,
(b) a flowing agent; and
(c) an agent selected from the group consisting of a sun protecting agent, an insect repelling agent, and a combination thereof,
in a carrier wherein the sanitizing composition is substantially free of a drying alcohol and wherein the subject suffers from a skin irritation selected from the group consisting of dry skin, itchy skin, red skin, bumpy skin, scaly skin, swollen skin and a dermal inflammatory condition selected from the group consisting of dermatitis, allergic contact dermatitis, seborrheic dermatitis, radiation dermatitis, psoriasis, pruritus, eczema, urticaria, angioedema, and combinations thereof,
wherein the sanitizing composition comprises less than 1% ethyl alcohol by weight of the total composition.

2. The method of claim 1, wherein the flowing agent is selected from the group consisting of magnesium stearate, calcium stearate, zinc state, stearic acid and fumed silica.

3. The method of claim 1, wherein the flowing agent is hydrophilic fumed silica.

4. The method of claim 1, wherein the sanitizing composition further comprises a component selected from the group consisting of a humectant, a lubricant, a carrier, a pharmaceutically active agent, a color, a fragrance, a preservative, an antioxidant, a chelator, a neutralizer, an amino acid, an anti-inflammatory agent, an anti-irritant, a binder, a stabilizer, an emollient, an emulsifier, a surfactant, a foaming agent, an essential oil, a plant/botanical extract, a skin conditioning agent, a gelling agent, a humectant, an antioxidant, a vitamin, a mineral, a viscosity enhancer, a pH modifier, a protein, a peptide, a tactile enhancer, and combinations thereof, wherein the component is a non-skin irritating component.

5. The method of claim 4, wherein the sanitizing composition further comprises at least one of a viscosity enhancer, a pH modifier, a humectant, an antioxidant, an anti-inflammatory agent, a skin conditioning agent, an essential oil, a vitamin and a mineral.

6. The method of claim 1, wherein the sun protecting agent is substantially free of para-aminobenzoic acid (PABA), paraben and/or phthalate.

7. The method of claim 1, wherein the sun protecting agent is selected from the group consisting of zinc oxide, titanium oxide, octocrylene, octyl salicylate, avobenzone, ecamsule, octyl triazone, amiloxate, diethylhexyl butamido trizone, enzacamene, drometrizole trisiloxane, bemotrizinol, bisoctrizole and combinations thereof.

8. The method of claim 1, wherein the sanitizing composition has a sun protection factor (SPF) in a range from about 8 to 50.

9. The method of claim 1, wherein the insect repelling agent comprises at least one of citronella, citronella oil, lemon grass oil, limonene, rosemary oil, mint oil, geranium oil, soybean oil, vegetable oil, eucalyptus oil, lemon juice, tea tree oil, camphor oil and eucalyptol.

10. The method of claim 1, wherein the sanitizing composition is a gel, lotion, cream, liquid, spray, foam or mousse.

11. The method of claim 1, wherein the subject suffers from dry skin, itchy skin, red skin, bumpy skin, scaly skin, swollen skin and combinations thereof.

12. The method of claim 1, wherein the subject suffers from a dermal inflammatory condition selected from the group consisting of dermatitis, allergic contact dermatitis, seborrheic dermatitis, radiation dermatitis, psoriasis, pruritus, eczema, urticaria, angiodema and combinations thereof.

13. The method of claim 12, wherein the subject suffers from eczema.

14. The method of claim 1, wherein the method is an alternative to hand washing with conventional soaps or cleansing with alcohol-based sanitizers.

15. The method of claim 1, wherein the microbial infection is a superficial microbial skin infection on a skin surface of the subject.

16. A sanitizing composition comprising:
(a) a sanitizing effective amount of an agent selected from the group consisting of hypochlorous acid, sodium hypochlorite, calcium hypochlorite and combinations thereof;
(b) a flowing agent selected from the group consisting of magnesium stearate, calcium stearate, zinc state, stearic acid and fumed silica; and
(c) a sun protecting agent that is substantially free of para-aminobenzoic acid (PABA), paraben and/or phthalate,
in a carrier wherein the composition is substantially free of a drying alcohol.

17. A non-skin irritating sanitizing composition comprising:
(a) a sanitizing effective amount of an agent selected from the group consisting of hypochlorous acid, sodium hypochlorite, calcium hypochlorite and combinations thereof;
(b) an oil phase; and
(c) an aqueous phase,
wherein the composition is substantially free of a drying alcohol.

18. The sanitizing composition of claim 16, wherein the agent is hypochlorous acid; the oil phase is emulsifying wax, jojoba oil, cetyl alcohol, shea butter, dimethicone, or a combination thereof; and the aqueous phase is water, glycerin or a combination thereof.
